(19) United States
(12) Patent Application Publication
(10) Pub. No.: US 2025/0345298 A1
(43) Pub. Date: Nov. 13, 2025

(54) APOE4 CORRECTORS AND METHODS OF USE

(71) Applicant: Alzheon, Inc., Framingham, MA (US)

(72) Inventors: John Hey, Framingham, MA (US); Martin Tolar, Farmingham, MA (US)

(21) Appl. No.: 18/860,990

(22) PCT Filed: Apr. 28, 2023
(86) PCT No.: PCT/US2023/020345

§ 371 Date: Oct. 28, 2024

Related U.S. Application Data
(60) Provisional application No. 63/336,210, filed on Apr. 28, 2022.

(51) Int. Cl. A61K 31/185; A61K 47/64

(52) U.S. Cl. CPC A61K 31/185; A61K 47/64

(57) ABSTRACT

Provided herein is the use of tramiprosate, a prodrug of tramiprosate, a tramiprosate analog, a prodrug of a tramiprosate analog, a pharmaceutically acceptable salt of any of the foregoing, or an isotopically enriched form of any of the foregoing for treating an ApoE4-related, non-amyloid disease or condition.

### RELATED APPLICATIONS

This application claims the benefit of priority to U.S. Provisional Application No. 63/336,210, filed Apr. 28, 2022, the entire contents of which are incorporated herein by reference.

### BACKGROUND

Apolipoprotein E4 (apoE4) is the major genetic risk factor for late onset Alzheimer disease (AD), increasing the risk of developing AD at earlier ages of onset. Human ApoE has 299 amino acids with three common isoforms (apoE2, apoE3, and apoE4) that differ at residue 112 or 158. ApoE3 contains a cysteine at residue 112 and an arginine at residue 158, whereas apoE4 has arginine at both positions, and apoE2 has cysteine. These minor variations give rise to profound differences in the tertiary protein structure and thereby function. ApoE4 displays an intramolecular domain interaction between its amino- and carboxyl-terminal domains, leading to a compact or “closed” structure. Domain interaction in apoE4 is induced by Arg-112, which facilitates the formation of a salt bridge between Arg-61 in the amino-terminal domain and Glu-255 in the carboxyl-terminal domain. The Cys-112 residue in apoE2 and apoE3 weakens the domain interaction, resulting in more open structures. See FIG. 1. Disrupting apoE4 domain interaction with small molecules, so-called apoE4 structure correctors, converts apoE4 into an apoE3-like conformation and reverses the apoE4-specific detrimental effects. Most correctors known to date are highly hydrophobic small molecules that do not seem to have high specificity for ApoE4 and thus have the potential for unwanted off-target effects that have impeded development or limited their utility as leads for developing new therapeutics.

In addition to playing a role in Alzheimer's disease and other amyloid-related disorders, apoE is also involved in cholesterol homeostasis and lipid metabolism and the apoE4 isozyme appears to have adverse effects on lipid profiles and cardiovascular diseases. Thus, apoE4 plays an important contributory role in hypercholesterolemia, coronary heart disease, cardiovascular disease, cerebral atherosclerosis, small vessel disease, and blood-brain barrier leakage. In contrast, apoE3 does not cause the same adverse effects. ApoE4 has also been linked to increased risk of nephrotic syndrome, gallstone disease, breast cancer, above average susceptibility to infectious diseases, and sub-normal immune response. The risk for these aforementioned disorders is increased further in subjects that have two copies or alleles of apoE4 (homozygous) vs. one copy of apoE4 vs lower risk in apoE4 non carriers.

Tramiprosate (also chemically referred to as homotaurine) has been investigated in human clinical trials for the treatment of Alzheimer's disease (AD). Although tramiprosate was never approved, the trial results suggested efficacy in at least some patients and a valine prodrug of tramiprosate, ALZ-801, is currently in human clinical trials for AD in ApoE4 homozygous subjects. It is believed that tramiprosate and its prodrug act by binding to amyloid-beta multimers and prevent the formation of the oligomers that are directly neurotoxic and become the main component of plaque found in AD brains. Tramiprosate and ALZ-801 were both found to be safe in human subjects.

Despite the identification of some small molecule apoE4 correctors, there is still a need for more apoE4 correctors with therapeutic potential that are safe and well tolerated, and more specifically bind to apoE4 and cause a structural change or modification (i.e. “corrector” action) resulting in a more open structure mimicking apoE3 or apoE2.

### SUMMARY OF THE INVENTION

The present disclosure solves the problem set forth above by demonstrating that tramiprosate also binds directly to ApoE4 at a site that binds heparin and other glycosaminoglycans (amino acids 141-150) and interacts with amino acids Asp107 (See FIG. 2 and FIG. 3). The location of these interactions is proximal to Arg112 and are likely to disrupt the salt bridge between Arg61 and Glu255 that is induced by Arg112 (FIG. 1). This, for the first time, suggests that tramiprosate, as well as derivatives thereof, and prodrugs and salts of tramiprosate or its derivatives are useful to treat ApoE4-related that do not involve amyloid or beta-amyloid.

Provided herein, therefore are methods of using tramiprosate, as well as derivatives thereof, and prodrugs and salts of tramiprosate or its derivatives for treating ApoE4-related that do not involve amyloid or beta-amyloid.

### BRIEF DESCRIPTION OF THE DRAWINGS

FIG. 1 is a ribbon diagram of the structures of ApoE4 and ApoE3 and shows the role of Arg-112 in forming a slat bridge between Arg-61 and Glu-225 in ApoE4. In ApoE3, Cys-112 replaces Arg-112 and no salt bridge exists between Arg-61 and Glu-225, resulting in more open structures.

FIG. 2 illustrates the binding of tramiprosate to ApoE4 at a site that binds heparin and other glycosaminoglycans (amino acids 141-150) and the interactions of that binding with nearby amino acids.

FIG. 3 illustrates the interaction of the sulfonic acid group of tramiprosate via an additional salt bridge in a bidentate fashion to the side chain of Arg150 (2.78 Aand 3.07 Å), as well as interactions with Asp-151 and Asp-107.

### DETAILED DESCRIPTION

In one aspect, provided are methods of treating an ApoE4-related, non-amyloid disease or condition comprising the step of administering to a subject in need thereof an effective amount of an agent selected from tramiprosate, a prodrug of tramiprosate, a tramiprosate analog, a prodrug of a tramiprosate analog, a pharmaceutically acceptable salt of any of the foregoing, or an isotopically enriched form of any of the foregoing.

Also provided is an effective amount of an agent selected from tramiprosate, a prodrug of tramiprosate, a tramiprosate analog, a prodrug of a tramiprosate analog, a pharmaceutically acceptable salt of any of the foregoing, or an isotopically enriched form of any of the foregoing, for treating an ApoE4-related, non-amyloid disease or condition.

Also provided in the use of an effective amount of an agent selected from tramiprosate, a prodrug of tramiprosate, a tramiprosate analog, a prodrug of a tramiprosate analog, a pharmaceutically acceptable salt of any of the foregoing, or an isotopically enriched form of any of the foregoing, for the manufacture of a medicament for treating an ApoE4-related, non-amyloid disease or condition.

Further provided is a composition comprising an effective amount of an agent selected from tramiprosate, a prodrug of tramiprosate, a tramiprosate analog, a prodrug of a tramiprosate analog, a pharmaceutically acceptable salt of any of the foregoing, or an isotopically enriched form of any of the foregoing, or an isotopically enriched form of any of the foregoing, for treating an ApoE4-related, non-amyloid disease or condition.

“Tramiprosate” (homotaurine, 3-amino-1-propanesulfonic acid (3-APS), or Alzhemed™) refers to the compound having the following chemical structure:

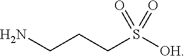

In one aspect, a “tramiprosate prodrug” or “prodrug of tramiprosate” includes, but is not limited to, a chemical compound that, after administration to a subject, is metabolized into tramiprosate. Tramiprosate prodrugs and analogs are known in the art and include those described in WO 2009/019534, WO 2017/027582, WO 2004/113275, WO 2006/085149, WO 1994/022437, WO 2000/064420, WO 1999/040909, WO 1999/059571, WO 2004/112762, and WO 2018/156845.

In one aspect, a tramiprosate prodrug refers to compounds having the formula: aa¹-aa²-NH—CH₂—CH₂—CH₂—S(O)₂—OH, wherein aa¹ is a natural or unnatural amino acid; aa² is a natural or unnatural amino acid, or is absent, and any hydrogen atom is optionally replaced with a deuterium atom. Other prodrugs include, but art not limited to, those having the formula:

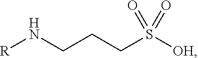

wherein R is -(AA¹)-(AA²)ₜ; AA¹ and AA² are each independently selected from alanine (Ala), cysteine (Cys), aspartic acid (Asp), glutamic acid (Glu), phenylalanine (Phe), glycine (Gly), histidine (His), isoleucine (Ile), lysine (Lys), leucine (Leu), methionine (Met), asparagine (Asn), proline (Pro), glutamine (Gln), arginine (Arg), serine (Ser), threonine (Thr), valine (Val), tryptophan (Trp), tyrosine (Tyr), β-alanine (β-ALA), and γ-aminobutyric acid (GABA); and t is 0 or 1. Additional prodrugs can be found in e.g., in WO 2015/143447.

In certain aspects, the tramiprosate prodrug is ALZ-801 (valyl-3-amino-1-propanesulfonic acid), represented by the following chemical structure

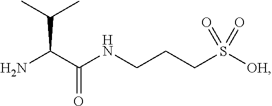

or a pharmaceutically acceptable salt thereof.

A “tramiprosate analog” refers to a chemical compound that is a modification of tramiprosate by one or more of: the addition of one or more additional substituents, the insertion or deletion of one or more methylene groups, the replacement of the SO₃H group with an isostere, and cyclization, while still maintaining the ability to bind to β-amyloid. In some embodiments, a tramiprosate analog is a compound described or disclosed in any of WO 2009/019534, WO 2017/027582, WO 2004/113275, WO 2006/085149, WO 1994/022437, WO 2000/064420, WO 1999/040909, WO 1999/059571, WO 2004/112762, and WO 2018/156845.

One example of such tramiprosate analogs include a compound having the formula:

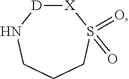

wherein D is carbonyl or a substituted methylene group; and X is selected from —O—, —NH—, or —S—.

Another example of tramiprosate analogs include a compound having the formula:

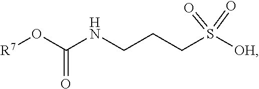

wherein R⁷ is is a substituted or unsubstituted group selected from C₁-C₁₂ alkyl, C₂-C₁₂ alkenyl, C₂-C₁₂ alkynyl, C₃-C₁₅ cycloalkyl, C₃-C₁₅ heterocycloalkyl, C₆-C₁₅ aryl, C₅-C₁₅ heteroaryl, C₇-C₁₂ arylalkyl, C₇-C₁₂ heteroarylalkyl, and combinations thereof.

Still another example of tramiprosated analogs include a compound having the formula:

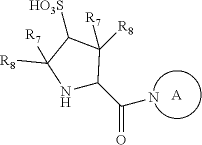

or the formula:

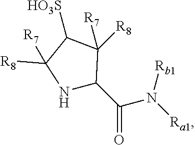

or a pharmaceutically acceptable salt of any of the foregoing, wherein:
Rₐ₁ is selected from the group consisting of hydrogen and C₁-C₃ alkyl optionally substituted with one or more hydroxyl;
R_{b1} is selected from the group consisting of hydrogen; C₁-C₃ alkyl substituted with one or more substituents independently selected from of carboxy, amino, optionally substituted heteroaryl, optionally substituted aryl, alkylthio, aminocarbonyl, hydroxy, dialkylamino, alkylamino, and arylalkylamino; cycloalkyl optionally substituted with amino; and heterocyclyl optionally substituted with amino;
each R⁷ and each R⁸ are hydrogen; and
ring A is selected from optionally substituted pyrrolidinyl, piperidinyl, morpholinyl, or piperazinyl.

The term “alkyl”, used alone or as a part of a larger moiety such as e.g., “haloalkyl”, means a saturated monovalent straight or branched hydrocarbon group having, unless otherwise specified, 1-10 carbon atoms and includes, for example, methyl, ethyl, n-propyl, isopropyl, n-butyl, sec-butyl, isobutyl, tert-butyl, n-pentyl, n-hexyl, n-heptyl, n-octyl, n-nonyl, n-decyl and the like.

The term “alkenyl”, used alone or as a part of a larger moiety such as e.g., “haloalkenyl”, means a monovalent group derived from a straight- or branched-chain aliphatic moiety having at least one carbon-carbon double bond having, unless otherwise specified 1-10 carbon atoms. Representative alkenyl groups include, but are not limited to, ethenyl (“vinyl”), propenyl (“allyl”), butenyl, 1-methyl-2-buten-1-yl, and the like.

The term “alkynyl”, used alone or as a part of a larger moiety such as e.g., “haloalkynyl”, means a monovalent group derived from a straight- or branched-chain aliphatic moiety having at least one carbon-carbon triple bond having, unless otherwise specified 1-10 carbon atoms. Representative alkynyl groups include, but are not limited to, ethynyl, 2-propynyl (“propargyl”), 1-propynyl, and the like.

The term “aryl” used alone or as part of a larger moiety as in “aralkyl” refers monocyclic and bicyclic carbon ring system having a total of five to 10 ring members, wherein at least one ring in the system is aromatic. The term “aryl” may be used interchangeably with the term “aryl ring”. In certain embodiments, “aryl” refers to an aromatic ring system which includes, but not limited to, phenyl, biphenyl, naphthyl, anthracyl and the like. In one embodiment, “aryl” is phenyl. It will be understood that when specified, optional substituents on an aryl group may be present on any substitutable position.

The term “heteroaryl” used alone or as part of a larger moiety as in “heteroarylalkyl”, refers to a 5- to 12-membered, fully aromatic ring system containing 1-4 heteroatoms selected from N, O, and S. The term “heteroaryl” may be used interchangeably with the terms “heteroaryl ring”, “heteroaryl group”, or “heteroaromatic”. A heteroaryl group may be mono- or bi-cyclic. Monocyclic heteroaryl includes, for example, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, and pyrazinyl. Bi-cyclic heteroaryls include groups in which a monocyclic heteroaryl ring is fused to one or more aryl or heteroaryl rings. Nonlimiting examples include indolyl, benzooxazolyl, benzooxodiazolyl, indazolyl, benzimidazolyl, benzthiazolyl, quinolyl, quinazolinyl, quinoxalinyl, pyrrolopyridinyl, pyrrolopyrimidinyl, pyrrolopyridinyl, thienopyridinyl, thienopyrimidinyl, indolizinyl, purinyl, naphthyridinyl, and pteridinyl. It will be understood that when specified, optional substituents on a heteroaryl group may be present on any substitutable position and, include, e.g., the position at which the heteroaryl is attached.

The term “heterocyclyl” means a 4- to 12-membered ring system, which is saturated or partially unsaturated (but not aromatic), containing 1 to 4 heteroatoms independently selected from N, O, and S. The terms “heterocycle”, “heterocyclyl”, “heterocyclyl ring”, “heterocyclic group”, and “heterocyclic moiety”, are used interchangeably herein. A heterocyclyl ring can be attached to its pendant group at any heteroatom or carbon atom that results in a stable structure. A heterocyclyl group may be mono- or bicyclic. Examples of monocyclic saturated or partially unsaturated heterocyclic groups include, without limitation, tetrahydrofuranyl, tetrahydrothienyl, tetrahydropyranyl, pyrrolidinyl, pyrrolidonyl, piperidinyl, oxazolidinyl, piperazinyl, dioxanyl, dioxolanyl, morpholinyl, dihydrofuranyl, dihydropyranyl, dihydropyridinyl, tetrahydropyridinyl, dihydropyrimidinyl, and tetrahydropyrimidinyl. Bi-cyclic heterocyclyl groups include, e.g., a heterocyclic ring fused to another unsaturated heterocyclic, cycloalkyl, aromatic or heteroaryl ring, such as for example, benzodioxolyl, dihydrobenzodioxinyl, 6,7-dihydro-5H-pyrrolo[2,1-c][1,2,4]triazolyl, 5,6,7,8-tetrahydroimidazo[1,2-a]pyridinyl, 1,2-dihydroquinolinyl, dihydrobenzofuranyl, tetrahydronaphthyridine, indolinone, dihydropyrrolotriazole, quinolinone, dioxaspirodecane. It will be understood that when specified, optional substituents on a heterocyclyl group may be present on any substitutable position and, include, e.g., the position at which the heterocyclyl is attached.

The term “cycloalkyl” refers to a monocyclic hydrocarbon or bicyclic hydrocarbon that is completely saturated.

The compounds described herein may have chiral centers and/or geometric centers (E- and Z-isomers). It will be understood that the present disclosure encompasses all stereoisomers and geometric isomers. Tautomeric forms of the compounds described herein are also part of the present disclosure.

The term “pharmaceutically acceptable salt” is a salt of a basic group (e.g., an amino group) or of an acidic group (e.g., a sulfonic acid) on the compounds described herein. Illustrative salts of a basic group include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucoronate, saccharate, formate, benzoate, glutamate, methanesulfonate, ethanesulfonate, benzenesulfonate, p-toluenesulfonate, camphorsulfonate, and pamoate (i.e., 1,1′-methylene-bis-(2-hydroxy-3-naphthoate)) salts. Illustrative salts of an acidic group include, but are not limited, to lithium, sodium, potassium, calcium, magnesium, aluminum, chromium, iron, copper, zinc, cadmium, ammonium, guanidinium, pyridinium, and organic ammonium salts.

“Pharmaceutically acceptable” refers to drugs, medicaments, inert ingredients etc., which the term describes, suitable for use in contact with the tissues of humans and lower animals without undue toxicity, incompatibility, instability, irritation, allergic response, and the like, commensurate with a reasonable benefit/risk ratio. In one aspect, pharmaceutically acceptable refers to a compound or composition that is approved or approvable by a regulatory agency of the Federal or state government or listed in the U.S. Pharmacopoeia or other generally recognized pharmacopoeia for use in animals and more particularly in humans.

Methods of administration can use an amount and a route of administration effective for treating or lessening the severity of a disease described herein. The exact amount required will vary from subject to subject, depending on the species, age, and general condition of the subject, the severity of the infection, the particular agent, its mode of administration, and the like. Provided compounds are preferably formulated in unit dosage form for ease of administration and uniformity of dosage. For example, provided compounds may be formulated such that a dosage of between 0.01-100 mg/kg body weight/day of the compound can be administered to a patient receiving these compositions. It will be understood, however, that the total daily usage of the compounds and compositions of the present disclosure will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts.

It should also be understood that a specific dosage and treatment regimen for any particular patient will depend upon a variety of factors, including age, body weight, general health, sex, diet, time of administration, rate of excretion, drug combination, the judgment of the treating physician, and the severity of the particular disease being treated.

As used herein the terms “subject” and “patient” may be used interchangeably, and means a mammal in need of treatment, e.g., companion animals (e.g., dogs, cats, and the like), farm animals (e.g., cows, pigs, horses, sheep, goats and the like) and laboratory animals (e.g., rats, mice, guinea pigs and the like). Typically, the subject is a human in need of treatment.

As used herein, the terms “treatment,” “treat,” and “treating” refer to reversing, alleviating, delaying the onset of, or inhibiting the progress of a disease, or one or more symptoms thereof, as described herein. In some embodiments, treatment may be administered after one or more symptoms have developed, i.e., therapeutic treatment. In other embodiments, treatment may be administered in the absence of symptoms. For example, treatment may be administered to a susceptible individual prior to the onset of symptoms (e.g., in light of a history of symptoms and/or in light of genetic or other susceptibility factors), i.e., prophylactic treatment. Treatment may also be continued after symptoms have resolved, for example to prevent or delay their recurrence.

ApoE4-related, non-amyloid diseases or conditions refer to diseases and conditions that are not known to be associated with amyloid deposits e.g., those which flourish in the presence of beta-amyloid plaques. ApoE4-related, non-amyloid diseases or conditions, include, but are not limited to hypercholesterolemia, coronary heart disease, cardiovascular disease, above average susceptibility to infectious diseases, sub-normal immune response, gallstones, cerebral atherosclerosis, small vessel disease, or blood-brain barrier leakage.

### EXEMPLIFICATION

### Example 1. Docking of Tramiprosate into an ApoE4 Crystal Model

A 1.70 Å resolution crystal structure of human ApoE4 (PDB Code: 1GS9) was utilized in the following studies to examine the possible binding modes of homotaurine. The 22kD fragment of ApoE4 was first processed via the Protein Preparation Wizard in Maestro (Schrodinger, “Maestro,” LLC, New York, NY, 2009) in which all crystallographic waters were removed, explicit hydrogen atoms added, and various conformational states of hydrogen bonding side chains were analyzed for their ability to form internal hydrogen bonds. The processed structure of ApoE4 was then “relaxed” via constrained minimization of all atoms present with the OPLS_2005 force field. The relaxed ApoE4 protein structure was used to generate a grid for pose evaluation via the Glide docking without any constraints. See R. A. Friesner, J. L. Banks, R. B. Murphy et al., “Glide: a new approach for rapid, accurate docking and scoring. 1. Method and assessment of docking accuracy,” J. Med. Chem. 2004, 47, 1739-1749. T. A. Halgren, R. B. Murphy, R. A. Friesner et al., “Glide: a new approach for rapid, accurate docking and scoring. 2. Enrichment factors in database screening,” J. Med. Chem. 2004, 47, 1750-1759.

The structure of homotaurine was docked using Glide in extra-precision mode (XP), with up to three poses saved for further optimization. Each of the docked poses was then energy-minimized in the bound state with the GBSA continuum solvation model (MM-GBSA). See Nu, H.; Kalyanaraman, C.; Irwin, J. J.; Jacobson, M. P. Physics based scoring of protein-ligand complexes: enrichment of known inhibitors in large-scale virtual screening. J. Chem. Inf. Model. 2006, 46, 243-253. In the energy minimization step, no constraints were applied to residues within 5 Å from the center of the homotaurine pose. The latter step was utilized in order to account for some degree of protein flexibility.

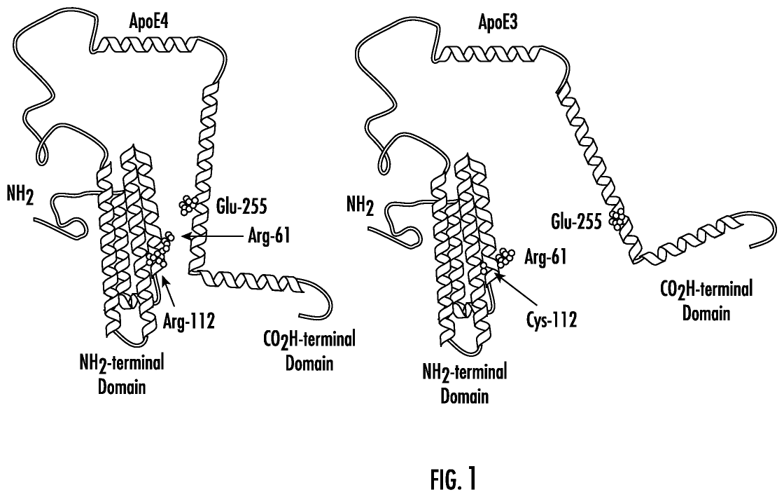

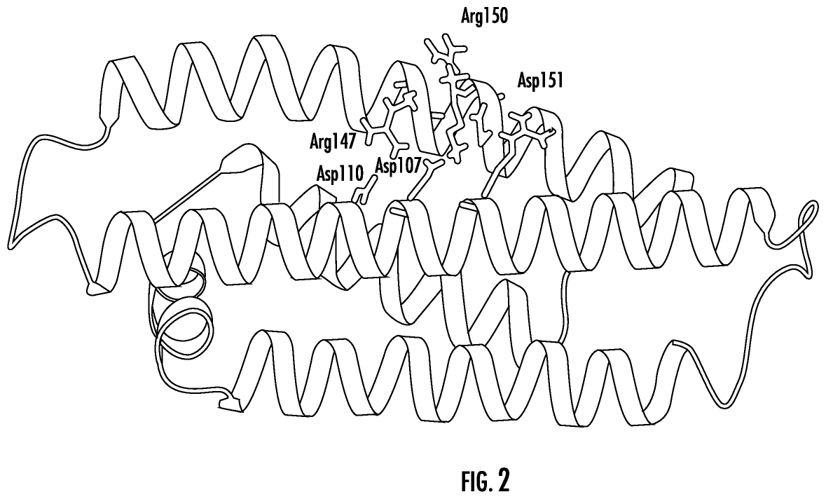

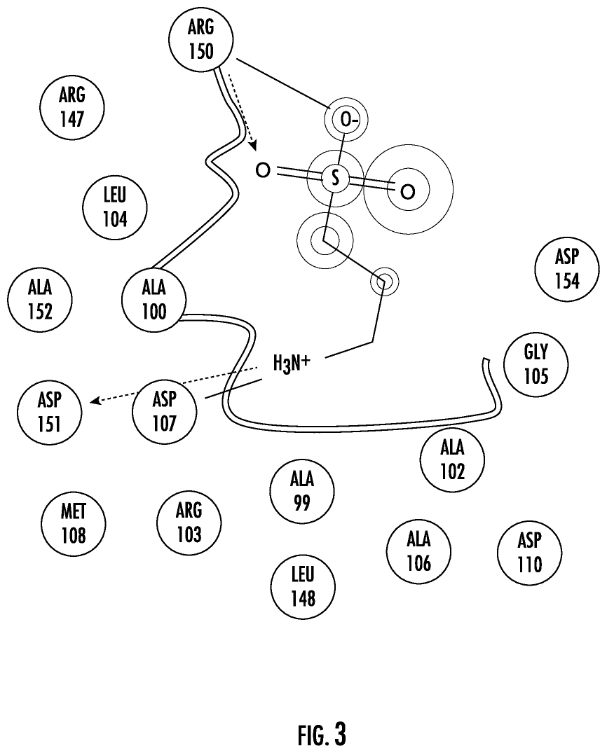

The top scoring poses were found to interact in the region of the heparin-binding domain of ApoE4 (residues 141-150) with the consensus sequence LRKLRKRLLR. See Datta, G.; D. W. Garber; B. H. Chung; M. Chaddha; N. Dashti; W. A. Bradley; S. H. Gianturco; G. M. Anantharamaiah. Cationic domain 141-150 of apoE covalently linked to a class A amphipathic helix enhances atherogenic lipoprotein metabolism in vitro and in vivo. J. Lipid Res. 2001, 42, 959-966. The primary interaction for homotaurine with ApoE4 is via pair of salt bridges between the protonated amine of homotaurine and the carboxylate side chains of Asp107 and Asp151 (3.05 Å and 2.80 Å, respectively). Additionally, the sulfonic acid group of homotaurine interacts via an additional salt bridge in a bidentate fashion to the side chain of Arg150 (2.78 Å and 3.07 Å). These interactions are illustrated in FIGS. 1-3.

While have described a number of embodiments of this, it is apparent that our basic examples may be altered to provide other embodiments that utilize the compounds and methods of this disclosure. Therefore, it will be appreciated that the scope of this disclosure is to be defined by the appended claims rather than by the specific embodiments that have been represented by way of example.

The contents of all references (including literature references, issued patents, published patent applications, and co-pending patent applications) cited throughout this application are hereby expressly incorporated herein in their entireties by reference. Unless otherwise defined, all technical and scientific terms used herein are accorded the meaning commonly known to one with ordinary skill in the art.

The invention claimed is:

1. A method of treating an ApoE4-related, non-amyloid disease or condition comprising the step of administering to a subject in need thereof an effective amount of an agent selected from tramiprosate, a prodrug of tramiprosate, a tramiprosate analog, a prodrug of a tramiprosate analog, a pharmaceutically acceptable salt of any of the foregoing, or an isotopically enriched form of any of the foregoing.

2. The method of claim 1, wherein the disease or condition is hypercholesterolemia, coronary heart disease, cardiovascular disease, above average susceptibility to infectious diseases, sub-normal immune response, gallstones, cerebral atherosclerosis, small vessel disease, or blood-brain barrier leakage.

3. The method of claim 1 or 2, wherein the agent is tramiprosate, or a prodrug of tramiprosate.

4. The method of claim 3, wherein the prodrug of tramiprosate is an amino acid prodrug of Formula (I): aa¹-aa²-NH—CH₂—CH₂—CH₂—S(O)₂—OH, wherein aa¹ is a natural or unnatural amino acid; aa² is a natural or unnatural amino acid, or is absent, and any hydrogen atom is optionally replaced with a deuterium atom.

5. The method of any one of claims 1-4, wherein the agent is formulated into an oral dosage form.


SEQUENCE LISTING (1 sequence)

SEQ ID NO: 1 - protein, 10 aa, synthetic construct
  REGION 1..10: note = Synthetic: consensus sequence
LRKLRKRLLR